(19) United States
(12) Patent Application Publication
(10) Pub. No.: US 2020/0237225 A1
(43) Pub. Date: Jul. 30, 2020

(54) WEARABLE PATIENT MONITORING SYSTEMS AND ASSOCIATED DEVICES, SYSTEMS, AND METHODS

(71) Applicant: Covidien LP, Mansfield, MA (US)

(72) Inventors: Paul S. Addison, Edinburgh (GB); Dominique Jacquel, Edinburgh (GB); Philip Smit, Hamilton (GB)

(21) Appl. No.: 16/750,497

(22) Filed: Jan. 23, 2020

Related U.S. Application Data
(60) Provisional application No. 62/797,519, filed on Jan. 28, 2019.

(51) Int. Cl. A61B 5/00; A61B 5/1171; A61B 5/11; A61B 5/0205; A61B 5/1455

(52) U.S. Cl. CPC A61B 5/0024; A61B 5/0077; A61B 5/1171; A61B 5/1128; A61B 5/022; A61B 5/02055; A61B 5/14552; A61B 5/746; A61B 2560/0214; A61B 5/6831

(57) ABSTRACT

The present technology relates to the field of medical monitoring. Patient monitoring systems and associated devices, methods, and computer readable media are described. In some embodiments, a patient monitoring system includes one or more sensors configured to capture first data related to a patient and a wearable wireless hub configured to receive the first data. In these and other embodiments, the patient monitoring system can include an image capture device configured to capture second data related to the patient. In these and still other embodiments, the patient monitoring system can determine one or more patient parameters based on the first and/or the second data. The patient monitoring system can monitor the one or more patent parameters and trigger an alert or alarm when a patient parameter abnormality is detected.

### CROSS-REFERENCE TO RELATED APPLICATIONS

This application claims priority to U.S. Provisional Patent Application Ser. No. 62/797,519, filed Jan. 28, 2019, the entire contents of which are incorporated herein by reference.

### FIELD

The present technology is generally related to wearable patient monitoring systems.

### BACKGROUND

Many conventional medical monitors require attachment of a sensor to a patient in order to detect physiologic signals from the patient and to transmit detected signals through a cable to the monitor. These monitors process the received signals and determine vital signs such as the patient's pulse rate, respiration rate, and arterial oxygen saturation. For example, a pulse oximeter is a finger sensor that can include two light emitters and a photodetector. The sensor emits light into the patient's finger and transmits the detected light signal to a monitor. The monitor includes a processor that processes the signal, determines vital signs (e.g., pulse rate, respiration rate, arterial oxygen saturation), and displays the vital signs on a display.

Other monitoring systems include other types of monitors and sensors, such as electroencephalogram (EEG) sensors, blood pressure cuffs, temperature probes, air flow measurement devices (e.g., spirometer), and others. Some wireless, wearable sensors have been developed, such as wireless EEG patches and wireless pulse oximetry sensors.

Video-based monitoring is a field of patient monitoring that uses one or more remote video cameras to detect physical attributes of the patient. This type of monitoring can also be called “non-contact” monitoring in reference to the remote video sensor(s), which does/do not contact the patient. The remainder of this disclosure offers solutions and improvements in this field.

### SUMMARY

The techniques of this disclosure generally relate to a patient monitoring system including a video monitoring system and a patient sensor that is in contact with the patient, both the video monitoring system and the sensor communicating patient data to a wearable wireless hub configured to attach to, be worn by, or be carried by the patient. In some aspects, a relay is configured to receive the first, sensor data and to transmit the data to the wearable wireless hub.

In another aspect, the disclosure provides video-based patient monitoring, wherein at least one region of interest (ROI) of a patient is defined, and wherein at least one image capture device captures two or more images of the ROI. A processor calculates a change in depth of at least one portion of the ROI within the two or more images.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a schematic view of a wearable patient monitoring system configured in accordance with various embodiments of the present technology.

FIG. 2 is a schematic view of a video-based patient monitoring system configured in accordance with various embodiments of the present technology.

FIGS. 3A and 3B are schematic views of patient monitoring systems configured in accordance with various embodiments of the present technology.

FIG. 3C is a flow diagram illustrating a routine for operating the systems of FIG. 3A and/or 3B.

FIG. 4 is a block diagram illustrating a patient monitoring system having a computing device, a server, and one or more image capture devices, and configured in accordance with various embodiments of the present technology.

FIG. 5 is a flow diagram illustrating a patient monitoring method for detecting and monitoring one or more patient parameters in accordance with various embodiments of the present technology.

FIG. 6 is a schematic view of a patient monitoring system configured in accordance with various embodiments of the present technology.

### DETAILED DESCRIPTION

The following disclosure describes patient monitoring devices, systems, and associated methods for detecting and/or monitoring one or more patient parameters, such as tidal volume, respiratory rate, minute volume, patient movement, temperature, blood pressure, heart rate, arterial oxygen saturation, and/or others. As described in greater detail below, devices, systems, and/or methods configured in accordance with embodiments of the present technology are configured to capture one or more images (e.g., a video sequence) of a patient or a portion of a patient (e.g., a patient's torso) within a field of view of a non-contact detector (e.g., an image capture device). The devices, systems, and/or methods can measure changes in depths of regions (e.g., one or more pixels or groups of pixels) in the captured images over time. Based, at least in part, on these measurements, the devices, systems, and/or methods can determine various respiratory parameters of a patient, including tidal volume, minute volume, respiratory rate, among others. In these and other embodiments, the device, systems, and/or methods can analyze the respiratory parameters and can trigger alerts and/or alarms when the devices, systems, and/or methods detect one or more breathing abnormalities.

Additionally or alternatively, devices, systems, and/or methods configured in accordance with embodiments of the present technology can include one or more sensors or probes associated with (e.g., contacting) a patient that can be configured to capture data (e.g., temperature, blood pressure, heart rate, arterial oxygen saturation, etc.) related to a patient. The devices, systems, and/or methods can transmit the captured data to a hub and/or central unit. In some embodiments, the devices, systems, and/or methods can analyze the captured data to determine and/or monitor one or more patient parameters. In these and other embodiments, the devices, systems, and/or methods can use the data captured by the one or more sensors or probes in conjunction with data captured using a non-contact detector. In these and still other embodiments, the devices, systems, and/or methods can trigger alerts and/or alarms when the devices, systems, and/or methods detect one or more patient parameter abnormalities.

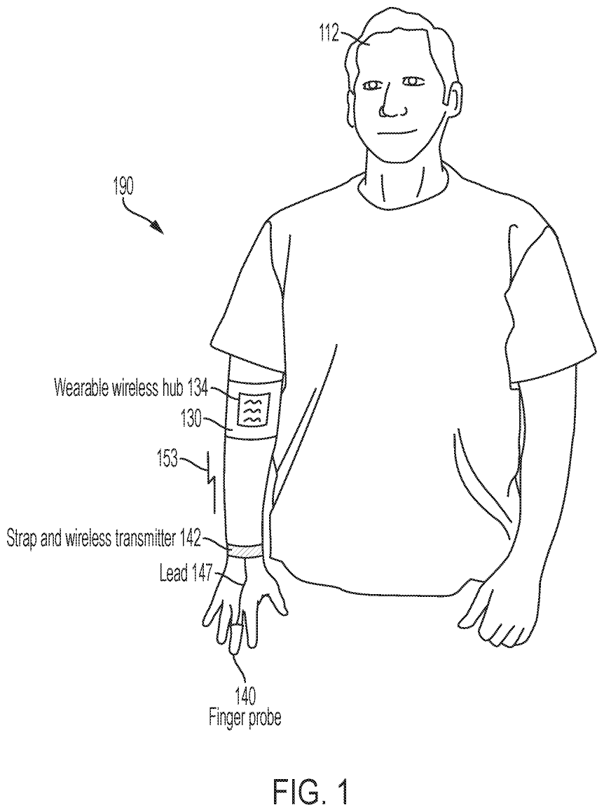

Specific details of several embodiments of the present technology are described herein with reference to FIGS. 1-6. Although many of the embodiments are described with respect to devices, systems, and methods for detection and/or monitoring of one or more parameters of a human patient, other applications and other embodiments in addition to those described herein are within the scope of the present technology. For example, at least some embodiments of the present technology can be useful for detection and/or monitoring of one or more parameters of other animals and/or in non-patients (e.g., elderly or neonatal individuals within their homes, individuals in a search and rescue or stranded context, etc.). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of a wearable patient monitoring system 190 configured in accordance with various embodiments of the present technology. As shown, the wearable system 190 includes a wireless hub 130, one or more sensors or probes 140, and a relay 142. In some embodiments, the wireless hub 130 can be a monitor with a screen 134 (e.g., to display various information, such as a power on/off button, one or more patient parameters, one or more alerts and/or alarms, etc.). The wireless hub 130 can be attached to, be worn, and/or otherwise be carried by a patient 112. For example, the wireless hub 130 can be attached to and/or worn by the patient 112 at the patient's upper arm, at the patient's belt, on the patient's wrist (e.g., as a watch and/or using a band), etc. In some embodiments, the wireless hub 130 can be sewn into the patient's clothing. In these and other embodiments, the wireless hub 130 can be a mobile device, such as a mobile phone, tablet, or laptop.

In the embodiments illustrated in FIG. 1, the one or more sensors 140 include a pulse oximeter attached to a finger of the patient 112. In these and other embodiments, the one or more sensors 140 can include other sensors in addition to or in lieu of the pulse oximeter, such as electrodes, temperature sensors, blood pressure cuffs, etc. The one or more sensors 140 can be used to perform various tests and/or to capture various information and data relating to the patient 112. For example, the one or more sensors 140 can be used to capture an electrocardiogram (ECG) signal and/or an electroencephalogram (EEG) signal of the patient. In these and other embodiments, the one or more sensors 140 can capture the patient's arterial oxygen saturation, temperature, blood pressure, and/or other patient parameters (e.g., systolic and diastolic pressure, heart rate, respiratory rate, average temperature, etc.).

Information captured by the one or more sensors 140 can be stored and/or processed by the one or more sensors 140 and/or by the wireless hub 130. For example, the one or more sensors 140 can store captured information and/or can locally process the captured information (e.g., to determine one or more patient parameters). In these and other embodiments, the one or more sensors 140 can transmit the raw, captured information and/or the locally processed data to the wireless hub 130. For example, the one or more sensors 140 can include a wireless transmitter (not shown) to transfer the data directly to the wireless hub 130 via a wired or wireless connection (not shown). In turn, the wireless hub 130 can store and/or process the information received from the one or more sensors 140 (e.g., to determine one or more patient parameters). In these and other embodiments, the wireless hub 130 can transfer the raw, captured information and/or processed data to a central unit (not shown), such as a central hospital station, via a wired or wireless connection (not shown).

Additionally or alternatively, as shown in FIG. 1, the one or more sensors 140 can transmit the captured information and/or the locally processed data to a relay 142 (e.g., a band attached to the patient 112) via one or more wired and/or wireless connections, such as over electrical leads or wires 147. In some embodiments, the relay 142 can store and/or process data received from the one or more sensors 140. In these and other embodiments, the relay 142 can include a wireless transmitter that can be used to transmit the captured information and/or the processed data to the wireless hub 130 via a wireless connection 153.

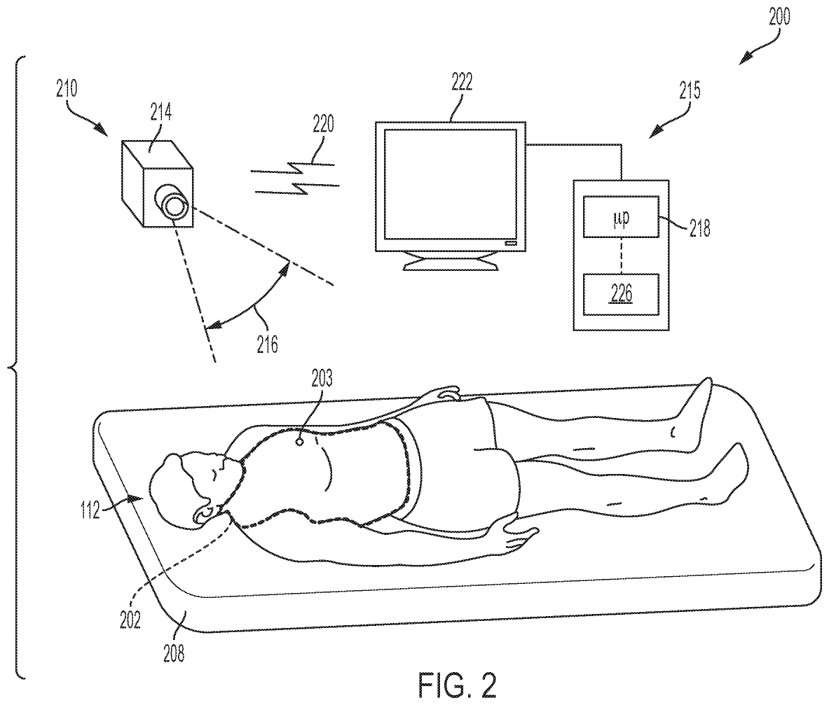

FIG. 2 is a schematic view of a patient 112 and a video-based patient monitoring system 200 configured in accordance with various embodiments of the present technology. The system 200 includes a non-contact detector 210 and a computing device 215. In some embodiments, the detector 210 can include one or more image capture devices 214, such as one or more video cameras. The non-contact detector 210 of the system 200 is placed remote from the patient 112. More specifically, the image capture device 214 of the non-contact detector 210 is positioned remote from the patient 112 in that it is spaced apart from and does not contact the patient 112. The image capture device 214 includes a detector exposed to a field of view (FOV) 216 that encompasses at least a portion of the patient 112.

The image capture device 214 can capture a sequence of images over time. The image capture device 214 can be a depth sensing camera, such as a Kinect camera from Microsoft Corp. (Redmond, Wash.). A depth sensing camera can detect a distance between the camera and objects within its field of view. Such information can be used, as disclosed herein, to determine that a patient 112 is within the FOV 216 of the image capture device 214 and/or to determine one or more ROI's to monitor on the patient 112. Once an ROI is identified, the ROI can be monitored over time, and the changes in depths of regions (e.g., pixels) within the ROI can represent movements of the patient 112 (e.g., associated with breathing). As described in greater detail in U.S. patent application Ser. No. 16/219,360 and U.S. Provisional Patent Application Ser. No. 62/779,964, those movements, or changes of regions within the ROI, can be used to determine various patient parameters, such as various breathing parameters, including tidal volume, minute volume, respiratory rate, etc. Those movements, or changes of regions within the ROI, can also be used to detect various patient parameter abnormalities, as discussed in greater detail in U.S. Provisional Patent Application Ser. Nos. 62/716,724 and 62/779,964. The various patient parameter abnormalities can include, for example, apnea, rapid breathing (tachypnea), slow breathing, intermittent or irregular breathing, shallow breathing, obstructed and/or impaired breathing, and others. The entire disclosures of U.S. patent application Ser. No. 16/219,360 and U.S. Provisional Patent Application Ser. Nos. 62/716,724 and 62/779,964 are incorporated herein by reference.

In these and other embodiments, the image capture device can be an RGB (red green blue) camera or an infrared camera. An RGB camera can detect slight color changes within its field of view. Such information can be used, as disclosed herein, to determine that a patient 112 is within the FOV 216 of the image capture device 214 and/or to determine one or more ROI's to monitor on the patient 112. Once an ROI is identified, the ROI can be monitored over time, and the changes in color of regions (e.g., pixels) within the ROI can represent various information related to the patient 112. As described in greater detail in U.S. patent application Ser. No. 16/188,969 those color changes can be used to detect optical signals associated with one or more medical devices, such as a pulse oximeter attached to the patient. Those color changes can also be used to determine and/or monitor various vital signs of the patient, including pulse rate, respiration rate, and arterial oxygen saturation, as discussed in greater detail in U.S. patent application Ser. Nos. 15/432,057 and 15/432,063. Additionally, or alternatively, as discussed in greater detail in U.S. Provisional Patent Application Ser. Nos. 62/685,485 and 62/695,244, those color changes can also be used in a surgical setting to monitor and/or assess blood flow in the ROI by detecting occlusions and/or monitoring pulsation, pulsation strength, and/or perfusion. The entire disclosures of U.S. patent application Ser. Nos. 16/188,969, 15/432,057, and 15/432,063 and U.S. Provisional Patent Application Ser. Nos. 62/685,485 and 62/695,244 are incorporated herein by reference.

In some embodiments, the system 200 can receive user input to identify a starting point for defining a ROI. For example, an image can be reproduced on a display 222 of the system 200 (or on the display 134 of the hub 130), allowing a user of the system 200 to select a patient 112 for monitoring (which can be helpful where multiple objects are within the FOV 216 of the image capture device 214) and/or allowing the user to select a point on the patient 112 from which a ROI can be determined (such as the point 203 on the chest of the patient 112). In other embodiments, other methods for identifying a patient 112, for identifying points on the patient 112, and/or for defining one or more ROI's can be used. For example, a user can select a patient 112 for monitoring and a point on a patient bed 208 (which can be helpful in defining one or more ranges of depths to be used in measurements taken by a non-contact detector).

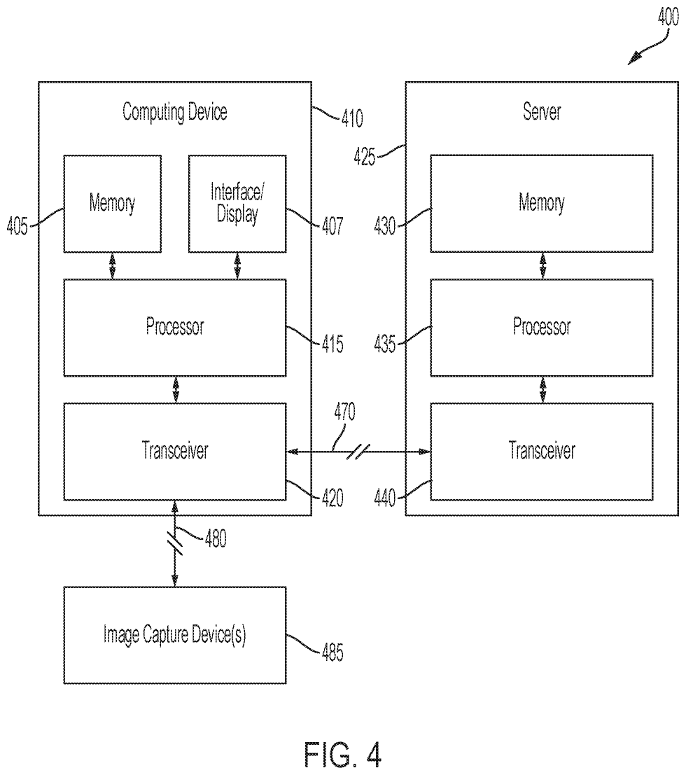

The images detected by the image capture device 214 can be sent to the computing device 215 through a wired or wireless connection 220. The computing device 215 can include a processor 218 (e.g., a microprocessor), the display 222, and/or hardware memory 226 for storing software and computer instructions. Sequential image frames of the patient 112 are recorded by the image capture device 214 and sent to the processor 218 for analysis. The display 222 can be remote from the image capture device 214, such as a video screen positioned separately from the processor 218 and the memory 226. Other embodiments of the computing device 215 can have different, fewer, or additional components than shown in FIG. 2. In some embodiments, the computing device 215 can be a server. In other embodiments, the computing device 215 of FIG. 2 can be additionally connected to a server (e.g., as shown in FIG. 4 and discussed in greater detail below). The captured images/video can be processed or analyzed at the computing device 215 and/or a server to determine a variety of parameters (e.g., tidal volume, minute volume, respiratory rate, etc.) of a patient.

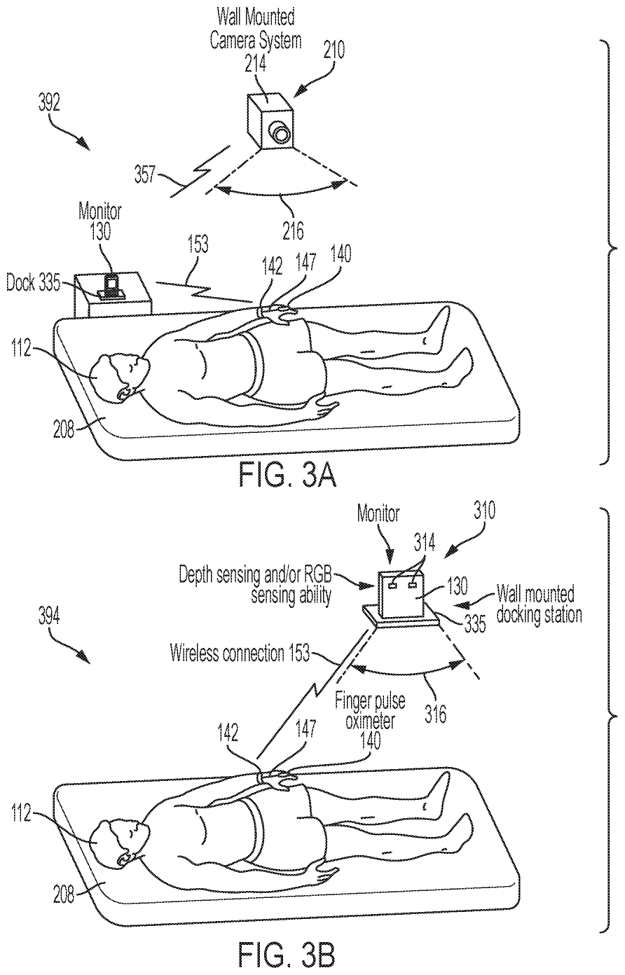

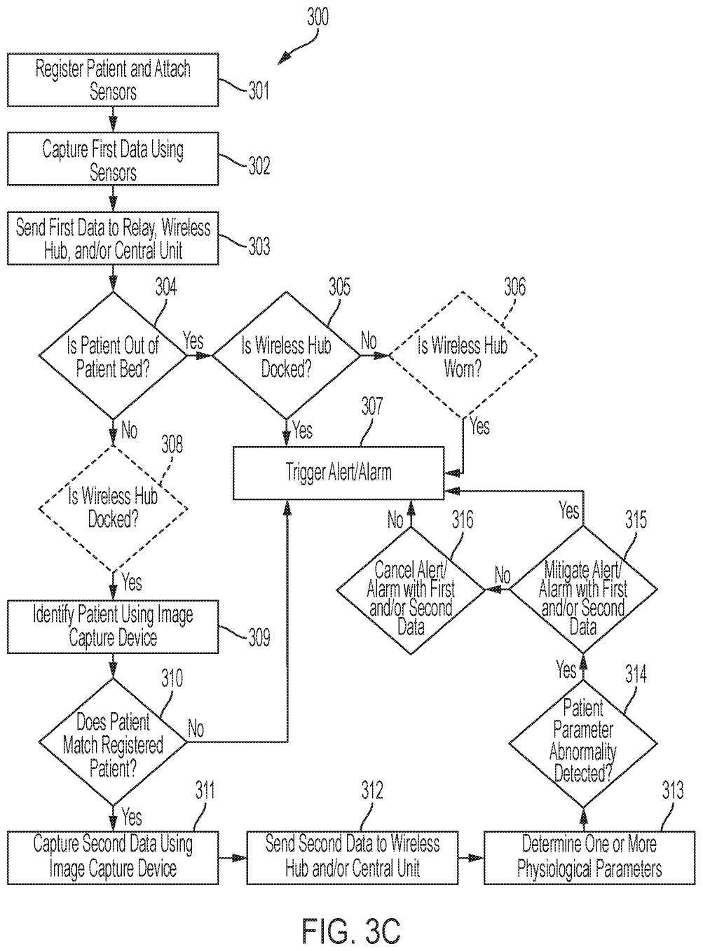

FIGS. 3A and 3B are schematic views of patient monitoring systems 392 and 394, respectively, configured in accordance with various embodiments of the present technology. The systems 392 and 394 can be similar to the patient monitoring system 190 illustrated in FIG. 1 in that the systems 392 and 394 can include a wireless hub 130, one or more sensors or probes 140, and a relay 142. Additionally or alternatively, the systems 392 and 394 can be similar to the system 200 illustrated in FIG. 2 in that the systems 392 and 394 can include one or more non-contact detectors 210 and 310, respectively, having one or more image capture devices 214 and 314, respectively.

Referring to FIG. 3A, the system 392 can be a combination of the system 190 shown in FIG. 1 and the system 200 shown in FIG. 2. In other words, the system 190 can be used in conjunction with the system 200 in some embodiments. The system 392 shown in FIG. 3A can further include a dock 335. The dock 335 can be configured to receive and/or charge the wireless hub 130. In the illustrated embodiment, the dock 335 can be placed on a bed side table adjacent a patient bed 208.

As shown in FIG. 3A, the image capture device 214 can be placed adjacent to and/or proximal a patient bed 208 such that the patient bed 208 is within a field of view (FOV) 216 of the image capture device 214. In some embodiments, the image capture device 214 can be configured to recognize when a patient 112 is in the patient bed 208. In these and other embodiments, the image capture device 214 can recognize the patient 112 (e.g., to ensure that a patient 112 registered with the wireless hub 130 corresponds to the patient 112 that the image capture device 214 recognizes within the patient bed 208). In these and still other embodiments, the image capture device 214 can be configured to capture image information (such as depth or RGB information) to detect, determine, and/or monitor physiological data (e.g., heart rate, respiratory rate, tidal volume, central apnea, obstructive apnea, motion activity, neck muscle activation, etc.) of the patient 112.

In some embodiments, the image capture device 214 can be configured to capture data when the wireless hub 130 is placed in the dock 335. Additionally or alternatively, the image capture device 214 can be configured to capture data when the image capture device 214 recognizes that a patient 112 is in the patient bed 208 and/or that the patient 112 corresponds with the patient 112 who is registered with the wireless hub 130. In these and other embodiments, the image capture device 214 can be configured to store and/or transmit data captured and/or processed to the wireless hub 130 (via a wired or wireless connection 357) and/or to a central unit (not shown), such as a central hospital station (not shown) via a wireless connection (not shown). In these and other embodiments, the wireless hub 130, the one or more sensors 140, and/or the relay 142 can be configured to send data to the image capture device 214 via various wired and/or wireless connections (not shown). In these and still other embodiments, data captured and/or processed can be processed at the image capture device 214, at the wireless hub 130, in the cloud, at the central station, and/or at other locations.

FIG. 3C is a flow diagram illustrating an exemplary routine 300 for operating the systems 392 and 394 of FIGS. 3A and 3B, respectively. At block 301, the routine 300 can register the wireless hub 130 to the patient 112 and attach the one or more sensors or probes 140 to the patient. At block 302, the routine 300 can capture first data using the sensors 140 (e.g., while the patient 112 is out of the patient bed 208), and the first data can be sent to the relay 142, to the wireless hub 130, and/or to a central unit (block 303). As the patient 112 leaves the patient bed 208 (block 304), the routine 300 can determine whether the wireless hub 130 is still placed in the dock 335 (block 305) and/or whether the patient 112 is wearing the wireless hub 130 (block 306). If the routine 300 determines that the patient 112 is out of the patient bed (block 304), the wireless hub 130 is still placed in the dock 335 (block 305), and/or the patient 112 is not wearing the wireless hub 130, the routine 300 can trigger an audio and/or visual alert/alarm (block 307) to remind the patient 112 to wear the wireless hub 130 while out of the patient bed 208 and/or while outside of the patient's room.

When the patient 112 returns to the patient bed 208 (block 304), the patient 112 can continue to wear the wireless hub 130 and/or can place the wireless hub 130 in the dock 335 (block 308). While the patient 112 is in the patient bed 208, the one or more sensors or probes 104 can continue to capture the first data (block 302) and/or send it to the relay 142, to the wireless hub 130, and/or to the central unit (block 303). As the patient 112 moves within the field of view 216 of the image capture device 214 (block 304) and/or as the patient 112 places the wireless hub 130 in the dock 335 (block 308), the routine 300, using the image capture device 214, (i) can identify the patient 112 (block 309), (ii) determine whether the patient 112 matches the patient 112 registered to the wireless hub 130 (block 310), and/or (iii) begin capturing second data (block 311). If the routine 300 determines at block 310 that the patient 112 identified at block 309 does not match the patient registered to the wireless hub 130 (block 301), the routine 300 can trigger an alert/alarm (block 307). On the other hand, if the routine determines at block 310 that the identified patient 112 (block 309) matches the patient 112 registered to the wireless hub 130 (block 301), the routine 300 can capture the second data (block 311) and send the second data to the wireless hub 130 and/or to the central unit (block 312).

Based at least in part on the first data and/or on the second data, the routine 300 can determine one or more physiological parameters of the patient 112 (block 313). If the routine 300 detects a patient parameter abnormality in the physiological parameters (block 314), the routine 300 can trigger an alert/alarm (block 307) to indicate that the patient 112 needs aid. In some cases, the routine 300 can detect a patient abnormality (block 314) when either the first data or the second data is corrupted or not available (e.g., when one of the sensors 140 attached to the patient 112 falls off the patient 112 while the patient 112 is in the patient bed 208). In these instances, the routine 300 can determine whether the patient 112 truly needs aid and/or whether the data is available from one of the other sources of information (block 315 and/or 316), such as from the image capture device 214. If the patient 112 doesn't need assistance and/or if the data is available from another source of information, the routine 300 can cancel or mitigate (e.g., downgrade) the alert/alarm at block 315 and 316, respectively. In this manner, the systems 392 and 394 (FIGS. 3A and 3B, respectively) can monitor a patient 112 regardless of whether a patient is in or out of the hospital bed 208 and/or regardless of whether meaningful data ceases to be available from a source of information.

Referring to FIG. 3B, the wearable patient monitoring system 394 can be used in a manner similar to the system 392 (FIG. 3A). The system 394 (FIG. 3B), however, differs from the system 392 in that the wireless hub 130 of the system 394 includes a non-contact detector 310. For example, the wireless hub 130 can include one or more image capture devices or cameras 314 (e.g., a depth sensing camera, an RGB camera, an infrared camera, and/or one or more other image capture devices). Thus, when not worn or carried by a patient 112, the image capture device(s) 314 of the wireless hub 130 can be placed adjacent and/or proximal to a patient bed 208 such that the patient bed 208 is within a field of view (FOV) 316 of the image capture device(s) 314. For example, the wireless hub 130 can be placed into a wall-mounted dock 335 configured to receive and/or charge the wireless hub 130. In these and other embodiments, the wireless hub 130 can be self-standing such that it can be placed on a table, shelf, or other surface. In some embodiments, the image capture device(s) 314 can be configured to capture data when the wireless hub 130 is placed into the dock 335. In these and other embodiments, the image capture device(s) 314 can be configured to operate similar to the non-contact detector 210 and the image capture device 214 illustrated in FIGS. 2 and 3A. In this manner, the system 394 can provide greater flexibility as the system 394 can be taken and/or set up anywhere the patient 112 is not mobile to capture data using the image device 214.

FIG. 4 is a block diagram illustrating a patient monitoring system 400 (e.g., the wearable patient monitoring system 190 shown in FIG. 1, the video-based patient monitoring system 200 shown in FIG. 2, the patient monitoring system 392 shown in FIG. 3A, and/or the patient monitoring system 394 shown in FIG. 3B) having a computing device 410, a server 425, and one or more image capture devices 485, and configured in accordance with various embodiments of the present technology. In various embodiments, fewer, additional, and/or different components can be used in the system 400. The computing device 410 includes a processor 415 that is coupled to a memory 405. The processor 415 can store and recall data and applications in the memory 405, including applications that process information and send commands/signals according to any of the methods disclosed herein. The processor 415 can also (i) display objects, applications, data, etc. on an interface/display 407 and/or (ii) receive inputs through the interface/display 407. As shown, the processor 415 is also coupled to a transceiver 420.

The computing device 410 can communicate with other devices, such as the server 425 and/or the image capture device(s) 485 via (e.g., wired or wireless) connections 470 and/or 480, respectively. For example, the computing device 410 can send to the server 425 information determined about a patient from images and/or other data captured by the image capture device(s) 485 and/or one or more other sensors or probes. The computing device 410 can be the computing device 215 of FIG. 2 and/or the wireless hub 130 of FIGS. 1, 3A, and/or 3B. Accordingly, the computing device 410 can be located remotely from the image capture device(s) 485, or it can be local and close to the image capture device(s) 485 (e.g., in the same room). In various embodiments disclosed herein, the processor 415 of the computing device 410 can perform the steps disclosed herein. In other embodiments, the steps can be performed on a processor 435 of the server 425. In some embodiments, the various steps and methods disclosed herein can be performed by both of the processors 415 and 435. In some embodiments, certain steps can be performed by the processor 415 while others are performed by the processor 435. In some embodiments, information determined by the processor 415 can be sent to the server 425 for storage and/or further processing.

In some embodiments, the image capture device(s) 485 are remote sensing device(s), such as depth sensing video camera(s), as described above with respect to FIGS. 2-3B. In some embodiments, the image capture device(s) 485 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter wave scanning technology can be utilized to scan a patient, which can be used to define and/or monitor a ROI. Advantageously, such technologies can be able to “see” through clothing, bedding, or other materials while giving an accurate representation of the patient's skin facet. This can allow for more accurate measurements, particularly if the patient is wearing baggy clothing or is under bedding. The image capture device(s) 485 can be described as local because they are relatively close in proximity to a patient such that at least a part of a patient is within the field of view of the image capture device(s) 485. In some embodiments, the image capture device(s) 485 can be adjustable to ensure that the patient is captured in the field of view. For example, the image capture device(s) 485 can be physically movable, can have a changeable orientation (such as by rotating or panning), and/or can be capable of changing a focus, zoom, or other characteristic to allow the image capture device(s) 485 to adequately capture images of a patient and/or a ROI of the patient. In various embodiments, for example, the image capture device(s) 485 can focus on a ROI, zoom in on the ROI, center the ROI within a field of view by moving the image capture device(s) 485, or otherwise adjust the field of view to allow for better and/or more accurate tracking/measurement of the ROI.

The server 425 includes a processor 435 that is coupled to a memory 430. The processor 435 can store and recall data and applications in the memory 430. The processor 435 is also coupled to a transceiver 440. In some embodiments, the processor 435, and subsequently the server 425, can communicate with other devices, such as the computing device 410 through the connection 470.

The devices shown in the illustrative embodiment can be utilized in various ways. For example, either the connections 470 and 480 can be varied. Either of the connections 470 and 480 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 470 and 480 can be a dock where one device can plug into another device. In other embodiments, either of the connections 470 and 480 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. Other possible modes of wireless communication can include near-field communications, such as passive radio-frequency identification (RFID) and active RFID technologies. RFID and similar near-field communications can allow the various devices to communicate in short range when they are placed proximate to one another. In some embodiments, two or more devices in the patient monitoring system 400 can together create a dynamic mesh network that includes connections 470 and/or 480. In these and other embodiments, data captured by and/or received at one device of the system 400 may be sent to and/or through other devices of the system 400 (e.g., to reach the server(s)), hence improving wireless coverage. In these and still other embodiments, the various devices can connect through an internet (or other network) connection. That is, either of the connections 470 and 480 can represent several different computing devices and network components that allow the various devices to communicate through the internet, either through a hard-wired or wireless connection. Either of the connections 470 and 480 can also be a combination of several modes of connection.

The configuration of the devices in FIG. 4 is merely one physical system 400 on which the disclosed embodiments can be executed. Other configurations of the devices shown can exist to practice the disclosed embodiments. Further, configurations of additional or fewer devices than the devices shown in FIG. 4 can exist to practice the disclosed embodiments. Additionally, the devices shown in FIG. 4 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

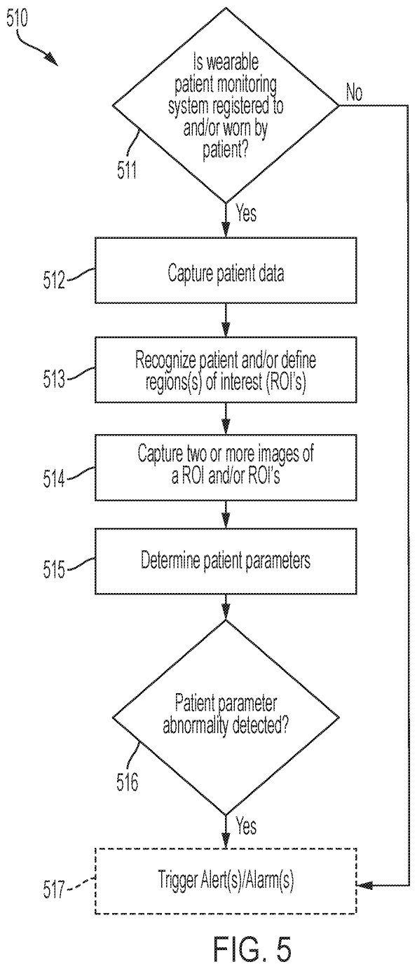

FIG. 5 is a flow diagram illustrating a patient monitoring routine 510 for determining and monitoring one or more patient parameters in accordance with various embodiments of the present technology. All or a subset of the steps of the routine 510 can be executed by various components or devices of one or more patient monitoring systems (e.g., the wearable and/or video-based patient monitoring systems described above) and/or users of the system(s) (e.g., a caregiver, a clinician, a patient, etc.).

The routine 510 can begin at block 511 by determining whether a wearable patient monitoring system is registered to and/or worn by a patient. In some embodiments, the routine 510 can determine whether a wearable patient monitoring system is registered to a patient by determining whether a wearable wireless hub is registered to a patient and/or whether the wireless hub is receiving data from one or more sensors or probes associated with (e.g., contacting) the patient. In these and other embodiments, the routine 510 can determine whether a wearable patient monitoring system is worn by the patient (i) by determining whether the wearable patient monitoring system is receiving data from one or more sensors or probes associated with the patient, (ii) by detecting motion data associated with the wireless hub (e.g., when the wireless hub is not docked), and/or (ii) by identifying the patient and/or the wireless hub within a field of view (FOV) of an image capture device. In some embodiments, the wireless hub can be configured to emit specific IR signals that the routine 510 can detect using an image capture device to locate the wireless hub within the field of view of the image capture device.

If the routine 510 determines that a wearable patient monitoring system is not registered and/or worn by a patient (e.g., that a wearable wireless hub of the wearable patient monitoring system is not receiving data from one or more sensors or probes associated with or contacting the patient), the routine 510 can proceed to block 518 to trigger one or more alerts and/or alarms. For example, the routine 510 can trigger an error message or sound to alert a user that a wearable patient monitoring system is not (e.g., properly) registered to a patient. In these and other embodiments, the routine 510 can trigger an alert and/or alarm to remind a user to wear the wearable patient monitoring system (e.g., to wear the wireless hub) and/or to notify a user that a patient is out of their hospital bed without their wireless hub. On the other hand, if the routine 510 determines that a wearable patient monitoring system is registered and/or worn by a patient, the routine 510 can proceed to block 512.

At block 512, the routine 510 can capture first patient data from a sensor in contact with the patient. In some embodiments, the routine 510 can capture patient data using one or more sensors or probes associated with (e.g., contacting) the patient. For example, the routine 510 can use one or more sensors or probes to capture an ECG signal, an EEG signal, a temperature signal, a heart rate signal, a respiratory rate signal, an average temperature signal, and/or one or more other physiological signals relating to the patient. In these and other embodiments, the routine 510 can capture patient data using one or more image capture devices. For example, the routine 510 can use an image capture device to detect a location of the patient, movement of the patient, whether the patient is awake or sleeping, and/or other data relating to the patient. In these and still other embodiments, the routine 510 can capture patient data by receiving data at a wearable wireless hub, at a central unit (e.g., a central hospital station), and/or at another location from the one or more sensors or probes, image capture device(s), and/or relay(s).

At block 513, the routine 510 can capture second patient data from the video system. For example, the routine 510 can recognize a patient within a field of view (FOV) of one or more image capture devices and/or define one or more regions of interest (ROI's) on the patient. In some embodiments, the routine 510 can recognize the patient by identifying the patient using facial recognition hardware and/or software of the image capture device(s). In these embodiments, the routine 510 can display the name of the patient on a display screen once the routine 510 has identified the patient. In some embodiments, the routine 510 can determine whether the recognized patient matches that patient registered to the wearable patient monitoring system at block 511, and/or can trigger one or more alerts and/or alarms if the patients do not match. In these and other embodiments, the routine 510 can recognize a patient within the FOV of the image capture device by determining a skeleton outline of the patient and/or by recognizing one or more characteristic features (e.g., a torso of a patient). In these and still other embodiments, the routine 510 can define one or more ROI's on the patient in accordance with the discussion above with respect to FIG. 2. For example, the routine 510 can define one or more ROI's on the patient using extrapolation from a point on the patient, using inferred positioning from proportional and/or spatial relationships with the patient's face, using parts of the patient having similar depths from the image capture device as a point, using one or more features on the patient's clothing, using user input, etc. In some embodiments, the routine 510 can recognize the patient and/or can define the ROI's when the routine 510 determines that a wearable wireless hub has been docked.

At block 514, the routine 510 can capture two or more images of one or more ROI's. In some embodiments, the routine 510 can capture the two or more images of the one or more ROI's by capturing a video sequence of the one or more ROI's. In these and other embodiments, the routine 510 can capture the two or more images of the one or more ROI's by capturing separate still images of the one or more ROI's. The routine 510 can capture the two or more still images at a rate faster than a period of the patient's respiration cycle to ensure that the two or more still images occur within one period of the patient's respiration cycle.

At block 515, the routine 510 can determine one or more patient parameters from the captured ROI's. In some embodiments, the routine 510 can determine one or more patient parameters using data captured at any of the blocks 512-514. For example, the routine 510 can determine a patient's respiratory rate, tidal volume, motion activity, temperature, blood pressure, arterial oxygen saturation, and/or other patient parameters using the captured data. In these and other embodiments, the routine 510 can determine the one or more patient parameters at the one or more sensors associated with (e.g., contacting) the patient, at a relay and/or wireless hub registered to the patient, at the one or more image capture devices, at a central unit (e.g., at a central hospital station), in the cloud, and/or at one or more other locations.

At block 516, the routine 510 can determine whether a patient parameter abnormality is detected based at least in part on data captured at any of the blocks 512-514 and/or on one or more patient parameters determined at block 515. For example, the routine 510 can detect that a patient is not breathing, is experiencing paradoxical breathing, is straining to breath, and/or is experiencing other breathing or patient parameter abnormalities (e.g., central apnea). In some embodiments, the routine 510 can use data captured with the one or more sensors or probes associated with (e.g., contacting) the patient in conjunction with data captured with the one or more image capture devices to determine whether the patient is experiencing a parameter abnormality. For example, the routine 510 can use data captured with an image capture device to mitigate a potential alert and/or alarm that would otherwise be triggered from a degraded signal captured with the one or more sensors or probes, or vice versa. In these embodiments, when one or more sensors or probes associated with the patient fall off the patient (e.g., while the patient is sleeping), the routine 510 can use data captured with the one or more image capture devices to monitor the patient. If the routine 510 does not detect a parameter abnormality using the data from the one or more image capture devices, the routine 510 can mitigate (e.g., not trigger or downgrade) an alert and/or alarm that otherwise would be triggered when the one or more sensors or probes fall off the patient (e.g., so as not to waken the patient).

As another example, the routine 510 can detect and monitor body motion using one or more image capture devices in conjunction with the one or more sensors or probes. In these embodiments, the routine 510 can detect when a patient is experiencing a seizure (e.g., characterized by significant muscle strain and/or regular jerking motion). In these and other embodiments, the routine can detect neck muscle activation using data captured with the one or more image capture devices in conjunction with an SpO₂ signal captured with the one or more sensors or probes to detect a patient's increased effort to breathe and/or to detect other obstructive or difficulty-of-breathing events. If the routine 510 detects a patient parameter abnormality, the routine 510 can proceed to block 518.

At block 517, the routine 510 can trigger one or more alerts and/or alarms. In some embodiments, the routine 510 can trigger one or more alerts and/or alarms at a wireless hub and/or at other components (e.g., a computer, an image capture device, a relay, a central unit, etc.) of a patient monitoring system. In these and other embodiments, the routine 510 can use a data buffer (e.g., a video buffer) such that when the routine 510 triggers an alert and/or alarm, the routine 510 can recall data in the buffer and/or save the data (e.g., in one or more logs for subsequent analysis). In these and still other embodiments, the routine 510 can associate a date and/or time stamp with the recalled and/or saved data to facilitate subsequent analysis and review of the data.

Although the steps of the routine 510 are discussed and illustrated in a particular order, the routine 510 illustrated in FIG. 5 is not so limited. In other embodiments, the routine 510 can be performed in a different order. In these and other embodiments, any of the steps of the routine 510 can be performed before, during, and/or after any of the other steps of the routine 510. For example, the blocks 513-514 can be performed before, during, after, and/or in lieu of the blocks 511 and/or 512. Moreover, a person of ordinary skill in the relevant art will readily recognize that the illustrated method can be altered and still remain within these and other embodiments of the present technology. For example, one or more steps of the routine 510 illustrated in FIG. 5 (e.g., the blocks 511 and/or 512) can be omitted and/or repeated in some embodiments.

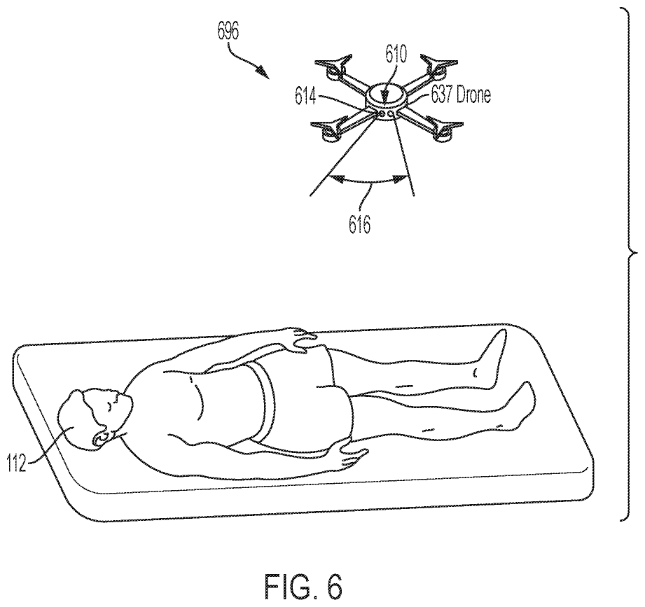

FIG. 6 is a schematic view of a patient monitoring system 696 configured in accordance with various embodiments of the present technology. As shown, the patient monitoring system 696 includes a drone 637 that has one or more non-contact detectors 610. In other embodiments, the patient monitoring system 696 can include other vehicles, such as other wheeled and/or legged vehicles, in addition to or in lieu of the drone 637. In the illustrated embodiment, the non-contact detector 610 includes an image capture device 614 (e.g., a depth sensing camera, an RGB camera, an infrared camera, and/or one or more other cameras) with a field of view (FOV) 616. In some embodiments, the non-contact detector 610 can be used to locate an individual (e.g., a person who has fallen, has collapsed, has become lost or stranded, and/or who otherwise needs to be monitored). Once located, the system 696 can monitor the patient in accordance with the discussion of FIGS. 1-5 above. For example, in the event of a disaster with limited access to an individual, the patient monitoring system 696 can be deployed and used to quickly respond to the situation (e.g., to locate the individual, to detect whether the individual is still alive, and/or to monitor the individual's vitals while help is on the way). In these and other embodiments, the patient monitoring system 696 can be further configured to deliver one or more sensors or probes (not shown) and/or relays (not shown) to the individual that can be attached to the individual and/or that can be used to detect and/or monitor one or more parameters of the individual. In these embodiments, the one or more sensors or probes and/or relays can communicate with (e.g., transmit captured data to) a processor (not shown) of the drone 637 and/or another remote device via one or more wired and/or wireless connections (not shown).

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A patient monitoring system, comprising:
a wearable wireless hub configured to receive from a sensor in contact with a patient first data related to the patient, and to process the first data to determine one or more patient parameters, wherein the wearable wireless hub is configured to attach to, be worn by, or be carried by the patient; and
a video monitoring system programmed to capture second data related to the patient and to communicate the second data to the wearable wireless hub, wherein the video monitoring system includes an image capture device, wherein the image capture device is configured along with the wearable wireless hub to provide identification of the patient associated with the wearable wireless hub, detection of motion of the patient or the wireless hub, or assessment of detected patient abnormality.

2. The patient monitoring system of claim 1, further comprising a relay configured to receive the first data from the one or more sensors and to transmit the first data to the wearable wireless hub.

3. The patient monitoring system of claim 2, wherein the relay includes a band configured to attach to the patient, and wherein the relay is configured to receive a first portion of the first data from the one or more sensors via an electrical wire or lead.

4. The patient monitoring system of claim 1, wherein the video monitoring system is programmed to:
define one or more regions of interest (ROI's) on a patient;
capture the second data related to the patient, wherein the second data includes two or more images of the ROI's; and
measure changes in depths of the ROI's across the two or more images of the ROI's.

5. The patient monitoring system of claim 1, wherein the image capture device includes a depth sensing camera, an RGB camera, and/or an infrared camera.

6. The patient monitoring system of claim 1, further comprising a dock configured to receive and charge the wearable wireless hub.

7. The patient monitoring system of claim 6, wherein the dock is configured to mount onto a wall.

8. The patient monitoring system of claim 1, wherein the wearable wireless hub includes the image capture device, wherein the video monitoring system includes the wearable wireless hub, and wherein the wearable wireless hub is configured to:
define one or more regions of interest (ROI's) on a patient;
capture second data related to the patient, wherein the second data includes two or more images of the ROI's; and
measure changes in depths of the ROI's across the two or more images of the ROI's.

9. The patient monitoring system of claim 1, further comprising a central unit configured to receive and/or process the first data captured using the one or more sensors and/or second data using the video monitoring system.

10. The patient monitoring system of claim 1, wherein the one or more sensors include a pulse oximeter, an electrode, a temperature sensor, and/or a blood pressure cuff.

11. The patient monitoring system of claim 1, wherein the wearable wireless hub includes a screen.

12. A method, comprising:
capturing first information using one or more sensors associated with a patient a wearable wireless hub configured to receive from a sensor in contact with a patient first data related to the patient, wherein the first information relates to one or more first physiological measurements of the patient;
capturing second information using an image capture device, wherein the second information relates to one or more second physiological measurements of the patient;
determining one or more patient parameters based, at least in part, on the first information; and
monitoring the one or more patient parameters to detect a first patient parameter abnormality, wherein the image capture device is configured along with the wearable wireless hub to provide assessment of detected patient abnormality.

13. The method of claim 12, further comprising:
determining at least one patient parameter based, at least in part, on the second information; and
monitoring the at least one patient parameter to detect a second patient parameter abnormality.

14. The method of claim 13, wherein (i) the one or more patient parameters include the at least one patient parameter and/or (ii) the first patient parameter abnormality is the same as the second patient parameter abnormality.

15. The method of claim 13, wherein capturing the second information using the image capture device include capturing the second information when a wearable wireless hub registered to the patient is docked and/or when the patient is sleeping.

16. The method of claim 13, further comprising recognizing the patient within a field of view of the image capture device, and wherein capturing the second information using the image capture device includes capturing the second information when the patient is recognized within the field of view.

17. The method of claim 13, wherein the patient is a first patient, and wherein the method further comprises:
identifying a second patient within a field of view of the image capture device; and
triggering an alert or alarm when the second patient is not the first patient.

18. The method of claim 13, further comprising triggering an alert or alarm when the first or the second patient parameter abnormality is detected.

19. The method of claim 13, further comprising mitigating a first triggered alert or alarm using the second information when the first patient parameter is detected and/or mitigating a second alert or alarm using the first information when the second patient parameter is detected.
